(19) United States
(12) Patent Application Publication
(10) Pub. No.: US 2012/0270758 A1
(43) Pub. Date: Oct. 25, 2012

(54) Degradable Surfactants, Including Degradable Gemini Surfactants, and Associated Methods

(75) Inventors: Rajesh K. Saini, Cypress, TX (US); Narongsak Tonmukayakul, Duncan, OK (US); Thomas D. Welton, Duncan, OK (US); Jason E. Bryant, Spring, TX (US); Sherry G. Gaskins, Lawton, OK (US)

(73) Assignee: Halliburton Energy Services, Inc., Houston, TX (US)

(21) Appl. No.: 13/533,362

(22) Filed: Jun. 26, 2012

Related U.S. Application Data
(62) Division of application No. 12/566,258, filed on Sep. 24, 2009.

(51) Int. Cl. C09K 8/04; C09K 8/34; C07C 229/26; C09K 8/60; C09K 8/68; C09K 8/64; C09K 8/32; C09K 8/00

(52) U.S. Cl. 507/119; 507/133; 507/131; 507/117; 507/139; 507/138; 507/103; 507/240; 507/220; 507/219; 507/224; 507/267; 507/265; 560/169

(57) ABSTRACT

A subterranean treatment fluid comprising a degradable gemini surfactant composition described by the following formula:

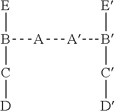

A and A′ are spacers that may be a hydrophobic group or a hydrophilic group. B is an ion and may contain a quaternary nitrogen, a sulfonate, or a phosphate. A, A′, B, B′, C, C, E and E′ may be an alkyl, an aryl, a sugar, an ester, an ether, and any combination thereof. D and D′ may be an aryl, a sugar, an ester, an ether, and any combination thereof. In some cases, A′, D′, and E′ may be optional.

### BACKGROUND

The present invention is related to surfactants, and more specifically, to degradable gemini surfactants including degradable gemini surfactants and their associated methods. Methods of use include subterranean operations, including those involving the placement of resin systems, formation of emulsions (e.g., emulsified acids, emulsified fracturing fluids, drilling fluids, etc.), and in the formation of surfactant gelled fluids.

Surfactants may be used in a variety of subterranean treatments. Such treatments include, but are not limited to, drilling, stimulation treatments (e.g., fracturing treatments, acidizing treatments, matrix acidizing, etc.), and completion operations (e.g., sand control treatments like gravel packing). As used herein, the term “treatment,” or “treating,” refers to any subterranean operation that uses a fluid in conjunction with a desired function and/or for a desired purpose.

Surfactants have been used heretofore in the art for many purposes, including stabilizing foams or emulsions, changing the wetability of surfaces, solubilizing certain materials, dewatering fluids, reducing the surface tension of fluids, increasing the viscosity of fluids, breaking emulsions, increasing compatibility of components of in fluids, corrosion inhibition, altering the zeta potential of a surface, enhancing viscoelastic and rheological properties of fluids, and/or aiding in the placement of treatment fluids in subterranean formations. The term “treatment,” or “treating,” does not imply any particular action by the fluid or any particular component thereof.

Surfactants, as that term is used herein, are thought of as surface-active agents, that are usually organic and whose molecules contain a hydrophilic group at one end and a lipophilic group at the other. Surfactants often act as wetting agents that are capable of reducing the surface tension of a liquid in which it is dissolved.

In the recovery of hydrocarbons, such as oil and gas, from subterranean formations, extensive use may be made of well treatment fluids such as drilling fluids, completion fluids, workover fluids, packer fluids, fracturing fluids, diverting fluids, acidizing fluids, conformance or permeability control fluids, and the like. In many cases, a significant component of these fluids is viscosifying agents that are based on either polymeric gelling agents (e.g., polysaccharides or synthetic polymers) or degradable gemini surfactants. These viscosifying agents contribute to and balance the viscosity of the fluids. The viscosified fluids may be crosslinked so as to increase the viscosity of the fluid to, for example, carry proppant particulates. Polymeric viscosified fluids have several drawbacks including the need of a breaker to reduce the viscosity to recover the fracturing fluid after treatment. Such polymers tend to leave a coating on surfaces within the formation that can negatively impact the conductivity of the formation.

Typical degradable gemini surfactants that are used to viscosify aqueous treatment fluids are predominantly cationic, such as quaternary ammonium halide salts or amphoteric/zwitterionic (such as betaine) derived from certain waxes, fats, and oils. These surfactants are typically used in conjunction with an inorganic water soluble salt such as potassium chloride or sodium chloride and an organic stabilizing additive such as sodium salicylate. Though there are many benefits of using surfactants to viscosify aqueous treatment fluids, they can suffer from several limitations.

First, a surfactant can interact adversely with the formation, which may result in a “wetting” of the formation. For example, a quaternary amine surfactant may change the surface wetability from water-wet to oil-wet, which may be undesirable. For instance, this change in wetability can be beneficial for the production of one phase (e.g., oil) and may not be better for the other phase (e.g., water), and there is a chance of the formation of block (oil or water) in the pores when the formation encounters the other phase. Additionally, a surfactant may interact with fluids in the formation to form relatively high viscosity emulsions; these can be problematic in that these emulsions may impair the permeability of the formation. Another problem that is encountered with surfactants is that they are generally not degradable and may persist in the formation for long periods of time.

An additional challenge can occur in the placement of resin and tackifier treatments. Surfactants may be used in such treatments to emulsify the resin or tackifier. This is generally viewed as an advantageous way of placing the resins and/or tackifiers because it makes the resin and/or tackifier easier to remove from the emulsion as it adheres to the formation. In the placement of resin treatments, for example, and especially aqueous resin treatments, the emulsifier or other surfactant present in the placement fluid may interfere with the resin or tackifier from adhering to the desired surfaces (e.g., sand, formation, etc.). Additionally, in some instances, surfactants may destabilize a coating (e.g., resin or tackifying agent) on a surface within a subterranean formation or a surface of a proppant particulate, for example, by forming surfactant micelles within the coating and/or making the coating less dense. In other instances, it may be desirable to deposit molecules of a surfactant on a surface within a subterranean formation and/or a surface of a proppant particulate, for example, when the proppant particulate is to be treated with certain aqueous tackifying agents. However, conventional surfactants may not distribute themselves evenly along the coating, which may leave certain portions of a subterranean formation or a proppant particulate insufficiently treated with the surfactant for particular subterranean operations.

### SUMMARY

The present invention is related to surfactants, and more specifically, to degradable gemini surfactants including degradable gemini surfactants and their associated methods. Methods of use include subterranean operations, including those involving the placement of resin systems formation of emulsions (e.g., emulsified acids, emulsified fracturing fluids, drilling fluids, etc.), and in the formation of surfactant gelled fluids.

In an embodiment, the present invention provides a method comprising: providing a subterranean treatment fluid that comprises a degradable gemini surfactant described by the following formula:

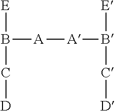

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional; and placing the subterranean treatment fluid in a subterranean formation.

In an embodiment, the present invention provides a method comprising: providing a fracturing fluid that comprises a base fluid, proppant particulates, and a viscosifying agent that comprises a degradable gemini surfactant described by the following formula:

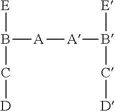

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional; and placing the fracturing fluid in a subterranean formation at a pressure sufficient to create or enhance at least one fracture therein.

In an embodiment the present invention provides a method comprising: providing a gravel pack fluid that comprises a base fluid, gravel particulates, and a viscosifying agent that comprises a degradable gemini surfactant described by the following formula:

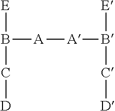

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional; and placing the gravel pack fluid in a subterranean formation so as to form a gravel pack neighboring a portion of the subterranean formation.

In another embodiment, the present invention provides a method comprising: providing a drilling fluid comprising a degradable gemini surfactant described by the following formula:

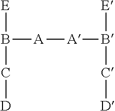

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional; and using the drilling fluid in a drilling operation to drill a well bore in a subterranean formation.

In another embodiment, the present invention provides a method comprising: providing an emulsified treatment fluid comprising an oleaginous phase, an aqueous phase, and an emulsifying agent that comprises a degradable gemini surfactant described by the following formula:

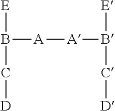

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional; and placing the emulsified treatment fluid in a subterranean formation.

In another embodiment, the present invention provides a subterranean treatment fluid comprising a degradable gemini surfactant composition described by the following formula:

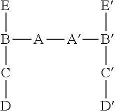

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional.

In yet another embodiment, the present invention provides a degradable gemini surfactant described by the following formula:

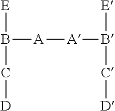

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional.

In yet another embodiment, the present invention provides an emulsified treatment fluid comprising an oleaginous phase, an aqueous phase, and an emulsifying agent that comprises a degradable gemini surfactant described by the following formula:

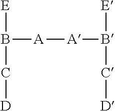

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof; and wherein A′, D, D′, E, and E′ are optional.

The features and advantages suitable for use in the present invention will be readily apparent to those skilled in the art. While numerous changes may be made by those skilled in the art, such changes are within the spirit of the invention.

### BRIEF DESCRIPTION OF THE DRAWINGS

These drawings illustrate certain aspects of some of the embodiments of the present invention, and should not be used to limit or define the invention.

FIG. 1 shows the schematic representation of a gemini surfactant structure.

FIG. 2 shows two different ways that the molecules can be attached by a spacer.

FIG. 3 shows the apparent viscosity of sample 5 (3 wt % of surfactant 10b and 0.6 wt % of sodium salicylate in water).

FIG. 4 shows the apparent viscosity of the sample 8 (3 wt % of surfactant 10b and 1.2 wt % of sodium salicylate in water).

FIG. 5 shows the apparent viscosity of sample 2 (2wt % of surfactant 6b and 2wt % of KCl in water).

FIG. 6 shows the apparent viscosity of sample 2, 5 and 8 at a fixed shear rate of 10 s⁻¹ as a function of temperature.

FIG. 7 shows the effect of temperature history on viscosity of sample 8 at a fixed shear rate of 10 s⁻¹.

FIG. 8 shows the temperature variation for surfactant gels tested for this application.

### DESCRIPTION OF PREFERRED EMBODIMENTS

The present invention is related to surfactants, and more specifically, to degradable gemini surfactants including degradable gemini surfactants and their associated methods. Methods of use include subterranean operations, including those involving the placement of resin systems, the formation of emulsified treatment fluids (e.g., emulsified acids, emulsified fracturing fluids, drilling fluids, etc.), and the formation of surfactant gelled fluids.

Of the many advantages of the present invention is the provision of degradable gemini surfactants that may be used to impart viscosity to an aqueous treatment fluid for use in suitable subterranean treatments. The degradable gemini surfactants suitable for use in the present invention are able to impart viscosity to aqueous systems, yet they can be easily degraded in the emulsion or gel form. Also, the degradable gemini surfactants suitable for use in the present invention are believed to not change the wetability of a subterranean formation in which they are used. Another advantage of the surfactants suitable for use in the present invention is that they can be used at low temperatures where other commercial surfactants cannot be degraded. Additionally, these surfactants viscosify water at a lower concentration than other commercially available surfactants. Also, the degradable gemini surfactants of the present invention may form a more stable emulsion under storage conditions for the delivery of a resin and/or tackifier that will then be released under usage conditions. Further, in the placement of resin treatments, the degradable gemini surfactants suitable for use in the present invention do not interfere with the resin or tackifier from adhering to surfaces. Additionally, use of the degradable gemini surfactants suitable for use in the present invention may aid in recovering treatments fluids by reducing undesired characteristics such as wetting or emulsion. Other benefits and advantages associated with the present invention will be apparent to one skilled in the art with the benefit of this disclosure.

Upon degradation, the degradable gemini surfactants may release a degradation product, such as an acid, that may be used to break a viscosified treatment fluid, degrade an acid-soluble component present in the subterranean formation, and/or to facilitate the setting of an acid-settable resin. In certain embodiments, the degradable gemini surfactants may also be used for any of a number of other functions, such as emulsifying agents, non-emulsifying agents, foaming agents, defoaming agents, viscosifying (or gelling) agents, dispersants, wetting agents, and the like. In some instances, the degradable gemini surfactants of the present invention include gemini surfactants, which are discussed below.

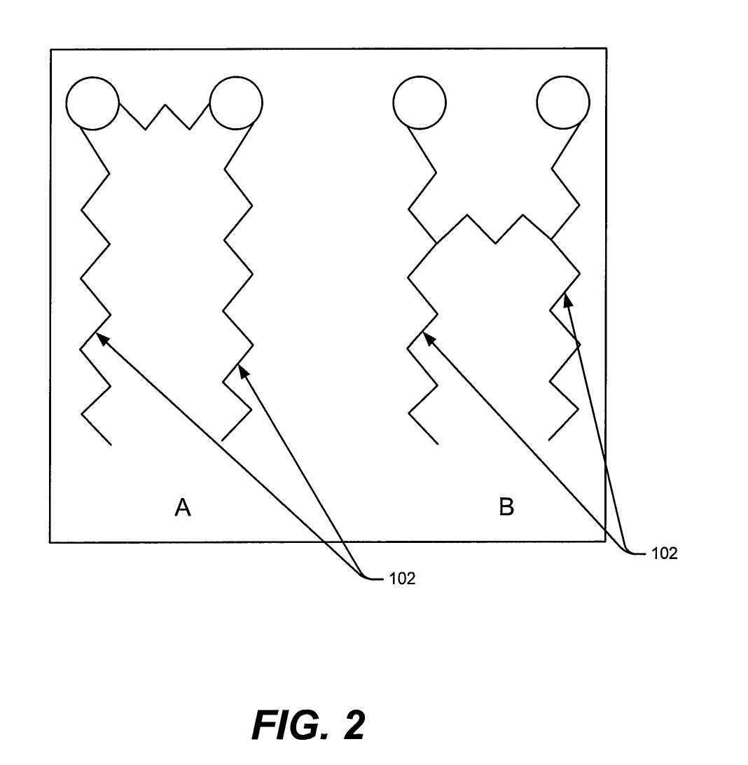

The degradable gemini surfactant compositions of the present invention comprise a degradable gemini surfactant. As used herein, the phrase “degradable gemini surfactant” refers to surfactant molecules, wherein the surfactant molecules contain one or more repeating units of degradable groups (e.g., as a spacer group or as a group in between the hydrophilic and hydrophobic groups), such as esters or other derivatives, for example, anhydrides, acetals, orthoesters, esteramides, ester ethers, ester carbonates, or ester urethanes as the degradable hydrophobic portion in the surfactant molecule attached to the hydrophilic portion, or as the degradable hydrophilic portion in the surfactant molecule attached to the hydrophobic portion. The term “gemini surfactant” as used herein refers to a surfactant that has at least two hydrophobic chains and two ionic or polar groups separated by a spacer. FIG. 2 depicts two ways that the molecules can be attached by a spacer.

Whereas conventional surfactant molecules (i.e., single chained amphiphile) are composed of a long hydrophobic hydrocarbon tail with an ionic or polar hydrophilic head, gemini surfactants are a family of surfactant compounds that contain two hydrophilic and two or more hydrophobic groups in the molecule. The two hydrophilic groups in the gemini surfactant are separated by a “spacer” or “linkage” containing one or more atoms. The spacer may be polar or non-polar in nature. These surfactants usually have better surface active properties than non-gemini surfactants of equal chain length.

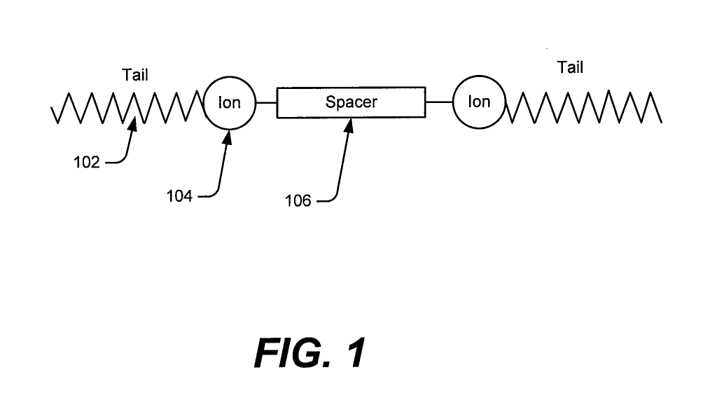

Gemini surfactants may have unusual structural features as illustrated in FIG. 1. Shown in FIG. 1 is a gemini surfactant that is an amphiphile made up of two hydrocarbon tails (102) and two ionic groups (104) linked by a spacer (106). The spacer can be attached directly to the identical ionic groups, in some instances, each of which in turn is bonded to an identical hydrocarbon tail. In alternative embodiments, the two identical amphiphile tails may be joined midway.

Because the degradable gemini surfactants have built into their structure a bond with limited stability, degradation of this bond should at least partially decrease the surface activity of the surfactants. In some embodiments, the degradable gemini surfactants may cleave at the juncture of the hydrophobic and hydrophilic unit for a particular surfactant molecule, which may result in the instantaneous disappearance of the surface activity for that surfactant molecule. As a result, the degradable gemini surfactants are capable of undergoing an irreversible degradation. The term “irreversible,” as used herein, means that the degradable gemini surfactant should degrade in situ (e.g., within a well bore), but should not reconstitute or reform in situ after degradation. The terms “degradation” and/or “degradable” refer to the conversion of materials into smaller components, intermediates, or end products by the result of hydrolytic degradation, biologically formed entities (e.g., bacteria or enzymes), chemical reactions, thermal reactions, or reactions induced by radiation.

All gemini surfactants possess at least two hydrophobic chains and two ionic or polar groups, and a great deal of variation exists in the nature of the spacers. The spacer length can be short (e.g., two methylene groups) or long (twelve methylene groups). Other examples of spacers are rigid groups (e.g., stilbene), polar groups (e.g., polyether), and nonpolar groups (e.g., aliphatic, aromatic). The ionic group can be positive or negative. The great majority of gemini surfactants have symmetrical structures with two identical polar groups and two identical chains. Some unsymmetrical gemini surfactants may be available. Generally, the structure can be adapted to make the surfactant more hydrophobic or more hydrophilic depending on the use. But increasing the hydrophobicity may make the molecule insoluble, whereas increasing hydrophilicity of the head group may impart solubility in water. Hydrophilic groups in the spacer also increase the aqueous solubility. Increasing the carbon number in the nonpolar chain may increase both the lipophilicity and surface activity with a decrease in the critical micellar concentration.

Additionally, gemini surfactants are believed to form micelles at lower concentrations than conventional surfactants of equal chain length. Generally, the addition of an effective surfactant lowers the surface tension of water until a critical micelle concentration (“CMC”) is reached. The CMC represents the point at which it is believed that individual surfactant molecules spontaneously aggregate into complex structures, including micelles, bilayer and vesicles. The micellization behavior of gemini surfactants is qualitatively different than that of conventional surfactants of equal chain length. The lower CMC is thought to be attributable to the increase in the number of hydrocarbon groups in the molecule. The CMC of a gemini surfactant is thought to be a non-monotonous function of the number of spacer hydrocarbon groups, which a maximum value around 4-6 methylene groups. Furthermore, for example, in the case of an ionic gemini surfactant, it is believed that the spacer reduces the intermolecular repulsion between head groups.

The type of aggregate formed is dependent on surfactant structure, temperature, ionic strength, and pH. Unlike conventional surfactants, which are believed to form spherical aggregates, gemini surfactants with short hydrophobic spacers are thought to form thread-like micelles and those with long spacers are believed to form rod-like micelles, especially when the spacer length is comparable to the tail length. The distribution of the head groups on the surface becomes inhomogeneous as linked head groups have a mutual distance different from that of the unlinked ones. The spacer gives the surfactant molecule an in-plane orientation, i.e., the combined head groups made of two monomeric heads and spacer is very anisotropic.

The degradable gemini surfactants useful in the present invention generally may be any suitable gemini surfactant. In certain embodiments, suitable gemini surfactant may be described by Formula I:

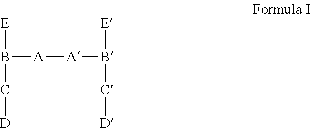

wherein A and A′ are spacers and may be a hydrophobic group or a hydrophilic group; B is an ion and may contain a quaternary nitrogen, a sulfonate, or a phosphate; and A, A′, B, B′, C, C′, D, D′, E and E′ may be an alkyl, an aryl, a sugar, an ester, an ether, and any combination thereof. The degradable unit may be between one or more of A, A′, B, C, D, and E. For example, a degradable unit may be located between D and E. Preferably, the degradable unit is not on the end. In certain embodiments, A′, D, D′, E, and E′ may be optional. In certain embodiments, B and B′ may be a cationic, anionic, nonionic, or zwitterionic moiety.

It is believed that the gemini surfactants of the present invention will also viscosify an aqueous fluid at lower concentrations than conventional surfactants or other degradable gemini surfactants. It is also believed that the spacer length can affect the viscosifying properties of a gemini surfactant. For example, a gemini surfactant with a spacer length of 2 is believed to have different viscosifying (also referred to as gelling) properties than a gemini surfactant with a spacer length of 5.

The degradability of the degradable gemini surfactants used in the present invention depends, at least in part, on the structure of the hydrophobic portion or the hydrophilic portion. For instance, the presence of hydrolyzable and/or oxidizable linkages often yields a degradable gemini surfactant that will degrade as described herein. The rates at which such surfactants degrade may be dependent on the type of repetitive unit, composition, length, hydrophilicity, hydrophobicity, and other additives that may be present. Other factors that may affect the degradation rate include the length of the hydrophilic tail and/or they hydrophobic tail. Also, the environment to which the degradable gemini surfactant is subjected may affect how it degrades, e.g., temperature, oxygen, microorganisms, enzymes, pH, and the like. Upon degradation, the degradable gemini surfactants may release a desirable degradation product, such as an acid that may be used in other applications downhole such as to degrade an acid-soluble component present in the subterranean formation, and/or to facilitate the setting of an acid-settable resin.

Among other things, degradation of the degradable gemini surfactants of the present invention may be sensitive to pH. For example, degradable gemini surfactants comprising an aliphatic polyester hydrophobic portion degrade rapidly at a higher pH (e.g., about 9 to about 14) and may be most stable at a pH of about 6. On the other hand, degradable gemini surfactants comprising a poly(orthoester) hydrophobic portion are stable at the higher pHs, but poly(orthoesters) may degrade at pHs of about 8 or less. With an increase in temperature, the hydrolysis of the surfactant should become faster.

The function that a particular degradable gemini surfactant of the present invention may perform depends on a variety of factors. These factors may include, but are not limited to, the choice of the hydrophobic and hydrophilic portions and the relative amounts thereof, and the presence of any cationic, anionic, non-ionic, amphoteric, or Zwitterionic groups. For example, whether an oil-in-water or water-in-oil emulsion is formed may be determined by the relative hydrophobicity of the degradable unitor tail and the hydrophilicity of the hydrophilic unit or head group. The hydrophilic/lipophilic balance (“HLB”) of the surfactant may provide a quantitative prediction of whether the surfactant will facilitate the formation of an oil-in-water or water-in-oil emulsion. HLB is a well known system that can be determined from the chemical formula of the surfactant using empirically determined group numbers.

By varying at least some of the above-listed factors, the specific properties of the degradable gemini surfactants such as solubility, wetability, emulsifying, foaming, antifoaming, cloud point, gelling, solubilizing agent, and the like may be varied. For example, where used as an emulsifying agent, a degradable gemini surfactant having an HLB of from about 3 to about 6 may be suitable to stabilize a w/o emulsion. In other embodiments, where used as an emulsifying agent, a degradable gemini surfactant having an HLB of from about 8 to about 18 may be suitable to stabilize an o/w. Those of ordinary skill in the art, with the benefit of this disclosure, will be able to determine the appropriate degradable gemini surfactants to use for a particular application.

The degradable gemini surfactants should be suitable for use at temperatures that they will encounter during subterranean operations, for as long a time period as maintenance of their surface activity is desired for the particular end use. Generally, the rates of degradation should increase with increasing temperature. At higher bottom-hole temperatures (e.g., greater than or equal to about 150° C.) certain degradable gemini surfactants, such as those having ester carbonates in the backbone, may be suitable for use. One of ordinary skill in the art, with the benefit of this disclosure, should be able to determine the appropriate degradable gemini surfactant to use based on, among other things, the particular bottom-hole temperatures that may be encountered.

The degradable gemini surfactant compositions suitable for use in the present invention may be used in any suitable treatment fluid for subterranean operations. Examples include consolidation fluids for the placement of resin and/or tackifier systems, emulsified treatment fluids (e.g., emulsified acids for stimulation treatments, emulsified fracturing fluids, emulsified drilling fluids, etc.), foamed fluids, and surfactant gelled fluids. Depending on the nature of the subterranean treatment fluid in which the degradable gemini surfactant composition is used, the fluid may contain additional additives suitable for use in the application in which the fluid will be used. When considering what additional components to include in an embodiment of the subterranean treatment fluids of the present invention, one should consider any potential negative interactions between the additional component and the degradable gemini surfactant composition.

In some instances, the degradable gemini surfactant compositions of the present invention may be used to viscosify treatment fluids. Viscosified treatment fluids comprising the degradable gemini surfactant compositions of the present invention may be used in any suitable subterranean application. Examples include fracturing, gravel packing, fluid loss control pills, and drilling applications.

In another embodiment, the present invention provides a method of viscosifying a treatment fluid comprising: providing a treatment fluid that comprises an aqueous base fluid and a degradable gemini surfactant composition of the present invention, wherein the treatment fluid has a first viscosity; and allowing the viscosity of the treatment fluid to increase to a second viscosity that is greater than the first viscosity as a result of the presence of the degradable gemini surfactant composition in the fluid.

The degradable gemini surfactant composition, in these embodiments, should be present in a treatment fluid in an amount sufficient to impart the desired viscosity (e.g., sufficient viscosity to divert flow, reduce fluid loss, suspend particulates, etc.) to the treatment fluid. In certain embodiments, the degradable gemini surfactant composition may be present in the treatment fluid in an amount in the range of from about 0.1% to about 20% by weight of the fluid. In certain embodiments, the degradable gemini surfactant composition may be present in an amount in the range of from about 1% to about 10% by weight of the fluid. In certain embodiments, the degradable gemini surfactant composition may be present in an amount of about 7% by weight of the fluid.

The aqueous base fluids used in the surfactant gelled fluids may comprise fresh water, saltwater (e.g., water containing one or more salts dissolved therein), brine, seawater, or combinations thereof. Generally, the water may be from any source, provided that it does not contain components that might adversely affect the stability and/or performance of the treatment fluids of the present invention. In certain embodiments, the density of the aqueous base fluid can be adjusted, among other purposes, to provide additional particle transport and suspension in the treatment fluids of the present invention. In certain embodiments, the pH of the aqueous base fluid may be adjusted (e.g., by a buffer or other pH adjusting agent), among other purposes, to reduce the viscosity of the treatment fluid (e.g., activate a breaker or other additive). In these embodiments, the pH may be adjusted to a specific level, which may depend on, among other factors, the types of degradable gemini surfactants, gelling agents, acids, and other additives included in the treatment fluid. One of ordinary skill in the art, with the benefit of this disclosure, will recognize when such density and/or pH adjustments are appropriate.

The surfactant gelled fluids used in methods of the present invention optionally may comprise any number of additional additives, including, but not limited to, salts, soaps, co-surfactants, carboxylic acids, acids, fluid loss control additives, gas, foamers, corrosion inhibitors, scale inhibitors, catalysts, clay control agents, biocides, friction reducers, antifoam agents, bridging agents, dispersants, flocculants, H₂S scavengers, CO₂ scavengers, oxygen scavengers, lubricants, viscosifiers, breakers, weighting agents, relative permeability modifiers, resins, particulate materials (e.g., proppant particulates), wetting agents, coating enhancement agents, combinations thereof and the like. A person skilled in the art, with the benefit of this disclosure, will recognize the types of additives that may be included in the treatment fluids for a particular application.

For example, the treatment fluids of the present invention optionally may comprise one or more salts, among other purposes, to modify the rheological properties (e.g., viscosity) of the treatment fluid. The salts may be organic or inorganic. Examples of suitable organic salts include but are not limited to aromatic sulfonates and carboxylates (such as p-toluene sulfonate, naphthalene sulfonate), hydroxynaphthalene carboxylates, salicylate, phthalate, chlorobenzoic acid, salicylic acid, phthalic acid, 5-hydroxy-1-naphthoic acid, 6-hydroxy-1-naphthoic acid, 7-hydroxy-1-naphthoic acid, 1-hydroxy-2-naphthoic acid, 3-hydroxy-2-naphthoic acid, 5-hydroxy-2-naphthoic acid, 7-hydroxy-2-naphthoic acid, 1,3-dihydroxy-2-naphthoic acid, 3,4-dichlorobenzoate, trimethylammonium hydrochloride and tetramethylammonium chloride. Examples of suitable inorganic salts include water-soluble potassium, sodium, and ammonium salts, (such as sodium chloride, potassium chloride, and ammonium chloride), calcium chloride, calcium bromide, magnesium chloride and zinc halide salts. Examples of treatment fluids comprising salts suitable for use in the present invention are described in U.S. patent application Ser. No. 10/800,478, the relevant disclosure of which is incorporated herein by reference. Any combination of the salts listed above also may be included in the treatment fluids of the present invention.

The salt may be present in any amount that imparts the desired stability and/or other rheological properties to a treatment fluid of the present invention. In certain embodiments, the salt may be present in an amount in the range of from about 0.05% to about 30% by weight of the treatment fluid. In certain embodiments, the salt may be present in an amount in the range of from about 0.1% to about 10% by weight of the treatment fluid. In certain embodiments, the salt may be present in an amount of about 5% by weight of the treatment fluid. The type and amount of salts suitable in a particular application of the present invention may depend upon a variety of factors, such as the type of degradable gemini surfactant present in the treatment fluid, the composition of the aqueous-base fluid, the temperature of the fluid, and the like. A person of ordinary skill, with the benefit of this disclosure, will recognize when to include a salt in a particular application of the present invention, as well as the appropriate type and amount of salts to include.

The treatment fluids optionally may comprise a co-surfactant, among other things, to facilitate the formation of and/or stabilize a foam, increase salt tolerability, and/or stabilize the treatment fluid. The co-surfactant may comprise any surfactant suitable for use in subterranean environments that does not adversely affect the treatment fluid. Examples of suitable co-surfactants include betaines (e.g., cocobetaine, cocoamidopropylbetaine), amine oxides, derivatives thereof, and combinations thereof. One of ordinary skill in the art will be able to determine which co-surfactants are best suited to the particular embodiments and applications of the compositions and methods described herein. For example, in some embodiments, the treatment fluids may be foamed by injection of a gas therein, wherein a co-surfactant (such as a cocobetaine) is included in treatment fluids to facilitate the formation of and/or stabilize the foam. In some embodiments, the co-surfactant may act to at least partially stabilize the treatment fluid. Generally, the co-surfactants may be present an amount sufficient to optimize the performance of the treatment fluid in a particular application, as determined by one of ordinary skill in the art.

The treatment fluids of the present invention also optionally may comprise one or more soaps, or substances that generate a soap when placed in solution (e.g., carboxylic acids). These soaps or substances that generate a soap when placed in solution are referred to herein as “soap components.” Among other purposes, the soap component may stabilize the treatment fluid and enhance its rheological properties (e.g., increase viscosity of the fluid), especially at higher temperatures (e.g., greater than about 200° F.). The term “soap” is defined herein to include salts of fatty acids. Examples of soaps that may be suitable for use in the present invention include sodium stereate, potassium stereate, ammonium stereate, sodium oleate, potassium oleate, ammonium oleate, sodium laurate, potassium laurate, sodium myristate, potassium myristate, sodium ricinoleate, potassium ricinoleate, sodium palmitate, potassium palmitate, calcium caprylate, sodium caprylate, potassium caprylate, and the like. In certain embodiments where it is desirable to include a soap in a treatment fluid of the present invention, one or more free carboxylic acids of a soap (e.g., fatty acids) may be placed in solution to generate a soap. Examples of carboxylic acids that may be suitable for this use include, but are not limited to, 4,7,10,13,16,19-docosahexaenoic acid, 4,7,10,13,16-docosapentaenoic acid, 5,8,11,14,17-eicosapentaenoic acid, 5,8,11,14-eicosatetraenoic acid, 5,8,11-eicosatrienoic acid, 6,9,12,15-octadecatetraenoic acid, 7,10,13,16,19-docosapentaenoic acid, 7,10,13,16-docosatetraenoic acid, 8,11,14,17-eicosatetraenoic acid, 8,11,14-eicosatrienoic acid, behenic acid, capric acid, caprylic acid, cis-11-docosenoic acid, cis-11-eicosenoic acid, cis-11-octadecenoic acid, cis-15-tetracosenoic acid, cis-4-decenoic acid, cis-4-dodecenoic acid, cis-4-tetradecenoic acid, cis-5-lauroleic acid, cis-5-tetradecenoic acid, cis-6-octadecenoic acid, cis-9-decenoic acid, cis-9-dodecenoic acid, cis-9-eicosenoic acid, cis-9-hexadecenoic acid, cis-9-tetradecenoic, cis-tetracosenoic acid, caprylic acid decenoic acid, dihydroxystearic acid, docosadienoic acid, docosahexaenoic acid, docosapentaenoic acid, dotriacontanoic acid, eicosadienoic acid, eicosanoic acid, eicosapentaenoic acid, eicosatetraenoic acid, eicosatrienoic acid, eicosenoic acid, erucic acid, heptadecanoic acid, heptadecenoic acid, hexacosanoic acid, hexadecadienoic acid, hexadecenoic acid, lauric acid, linoleic acid, linolenic, myristic acid, nonadecanoic acid, nonanoic acid, octacosanoic acid, octadecatetraenoic acid, octadecatrienoic acid, oleic acid, palmitic acid, pentadecanoic acid, pentadecenoic acid, pentatriacontanoic, ricinoleic acid, stearic acid, tetracosanoic acid, tetradecenoic acid, tetratriacontanoic acid, triacontanoic acid, tridecanoic acid, tritriacontanoic acid, combinations thereof, and the like.

The soap component also may be combination of fatty acids made from numerous sources including but limited to animal fats, marine fats, vegetable oils and fats, butter, canola oil, castor oil, coco butter coconut oil, corn oil, cotton seed oil, crambe oil, herrings, lard, linseed oil, menhaden, olive oil, palm kernel oil, peanut oil, palm oil, rape seed oil, safflower oil, sardines, soybean oil, sunflower oil, tall oil, tallow, tung oil, yellow grease, combinations thereof, and the like. Any combination of the soaps or free fatty acids listed above also may be included in the treatment fluids of the present invention. The type and amount of soap components suitable in a particular application of the present invention may depend upon a variety of factors, such as the type of degradable gemini surfactant present in the treatment fluid, the composition of the aqueous-base fluid, the temperature of the fluid, and the like. For example, certain types of soap components may be incompatible with certain components of the treatment fluid and/or produce undesirable characteristics in the fluid (e.g., reduced viscosity and/or stability).

The soap component may be present in any amount that imparts the desired stability and/or other rheological properties to the treatment fluid of the present invention. In certain embodiments, the soap component may be present in the treatment fluid in an amount in the range of from about 0.01% to about 10% by weight of the fluid. In certain embodiments, the soap component may be present in an amount in the range of from about 0.05% to about 2% by weight of the fluid. In certain embodiments, the soap component may be present in an amount of about 0.14% by weight of the fluid. A person of ordinary skill, with the benefit of this disclosure, will recognize when to include a soap component in a particular application of the present invention, as well as the appropriate type and amount of soap component to include.

In one embodiment, the present invention provides a method comprising: providing a treatment fluid that comprises an aqueous base fluid, and a degradable gemini surfactant composition of the present invention; and introducing the treatment fluid into a subterranean formation.

Consolidation treatment fluids suitable for use in the present invention for the placement of a resin and/or a tackifier may comprise an aqueous base fluid and a degradable gemini surfactant composition suitable for use in the present invention in addition to the chosen resin and/or tackifier. In alternative embodiments, the resin and/or the tackifier may be coated on proppant particulates or gravel particulates, if desired. Suitable additives may be included as well as recognized by those skilled in the art.

These consolidation fluids may be placed downhole to consolidate loose particulates within a formation that might otherwise be flowed back, which can be problematic. In one embodiment, the present invention provides a method of consolidating particulates in a subterranean formation that comprises: providing a consolidation fluid that comprises an aqueous base fluid, a degradable gemini surfactant of the present invention, and a resin and/or a tackifier; placing the consolidation fluid in a subterranean formation via a well bore; and allowing the consolidation fluid to consolidate at least a plurality of particulates in the formation.

Although any suitable resin, tackifier, or polymer system that may be used in consolidation applications downhole may be used in conjunction with the degradable gemini surfactants of the present invention, the following nonlimiting examples are given below.

Resins suitable for use in the consolidation fluids include all resins known in the art that are capable of forming a hardened, consolidated mass. Many such resins are commonly used in subterranean consolidation operations, and some suitable resins include two component epoxy based resins, novolak resins, polyepoxide resins, phenol-aldehyde resins, urea-aldehyde resins, urethane resins, phenolic resins, furan resins, furan/furfuryl alcohol resins, phenolic/latex resins, phenol formaldehyde resins, polyester resins and hybrids and copolymers thereof, polyurethane resins and hybrids and copolymers thereof, acrylate resins, and mixtures thereof Some suitable resins, such as epoxy resins, may be cured with an internal catalyst or activator so that when pumped down hole, they may be cured using only time and temperature. Other suitable resins, such as furan resins generally require a time-delayed catalyst or an external catalyst to help activate the polymerization of the resins if the cure temperature is low (i.e., less than 250° F.), but will cure under the effect of time and temperature if the formation temperature is above about 250° F., preferably above about 300° F. It is within the ability of one skilled in the art, with the benefit of this disclosure, to select a suitable resin for use in embodiments suitable for use in the present invention and to determine whether a catalyst is required to trigger curing.

Selection of a suitable resin may be affected by the temperature of the subterranean formation to which the fluid will be introduced. By way of example, for subterranean formations having a bottom hole static temperature (“BHST”) ranging from about 60° F. to about 250° F., two-component epoxy-based resins comprising a hardenable resin component and a hardening agent component containing specific hardening agents may be preferred. For subterranean formations having a BHST ranging from about 300° F. to about 600° F., a furan-based resin may be preferred. For subterranean formations having a BHST ranging from about 200° F. to about 400° F., either a phenolic-based resin or a one-component HT epoxy-based resin may be suitable. For subterranean formations having a BHST of at least about 175° F., a phenol/phenol formaldehyde/furfuryl alcohol resin may also be suitable.

Any solvent that is compatible with the chosen resin and achieves the desired viscosity effect is suitable for use in the present invention. Some preferred solvents are those having high flash points (e.g., about 125° F.) because of, among other things, environmental and safety concerns; such solvents include butyl lactate, butylglycidyl ether, dipropylene glycol methyl ether, dipropylene glycol dimethyl ether, dimethyl formamide, diethyleneglycol methyl ether, ethyleneglycol butyl ether, diethyleneglycol butyl ether, propylene carbonate, methanol, butyl alcohol, d'limonene, fatty acid methyl esters, and combinations thereof. Other preferred solvents include aqueous dissolvable solvents such as, methanol, isopropanol, butanol, glycol ether solvents, and combinations thereof Suitable glycol ether solvents include, but are not limited to, diethylene glycol methyl ether, dipropylene glycol methyl ether, 2-butoxy ethanol, ethers of a C₂ to C₆ dihydric alkanol containing at least one C₁ to C₆ alkyl group, mono ethers of dihydric alkanols, methoxypropanol, butoxyethanol, hexoxyethanol, and isomers thereof. Selection of an appropriate solvent is dependent on the resin chosen and is within the ability of one skilled in the art with the benefit of this disclosure.

Tackifying agents suitable for use in the present invention include non-aqueous tackifying agents; aqueous tackifying agents; and silyl-modified polyamides. In addition to encouraging the proppant particulates to form aggregates, the use of a tackifying agent may yield a propped fracture that experiences very little or no undesirable proppant flow back. The application of a tackifying agent to the proppant particulates may aid in the formation of aggregates that increase the ability of a smaller amount of proppant particulates to effectively hold open a fracture for production. Tackifying agents may be applied on-the-fly, applying the adhesive substance to the proppant particulate at the well site, directly prior to pumping the fluid-proppant mixture into the well bore.

One type of tackifying agent suitable for use in the present invention is a non-aqueous tackifying agent. A particularly preferred group of tackifying agents comprise polyamides that are liquids or in solution at the temperature of the subterranean formation such that they are, by themselves, non-hardening when introduced into the subterranean formation. A particularly preferred product is a condensation reaction product comprised of commercially available polyacids and a polyamine. Such commercial products include compounds such as mixtures of C₃₆ dibasic acids containing some trimer and higher oligomers and also small amounts of monomer acids that are reacted with polyamines. Other polyacids include trimer acids, synthetic acids produced from fatty acids, maleic anhydride, acrylic acid, and the like. Such acid compounds are commercially available from companies such as Witco Corporation, Union Camp, Chemtall, and Emery Industries. The reaction products are available from, for example, Champion Technologies, Inc. and Witco Corporation. Additional compounds which may be used as non-aqueous tackifying compounds include liquids and solutions of, for example, polyesters, polycarbonates and polycarbamates, natural resins such as shellac and the like. Other suitable non-aqueous tackifying agents are described in U.S. Pat. No. 5,853,048 issued to Weaver, et al. and U.S. Pat. No. 5,833,000 issued to Weaver, et al., the relevant disclosures of which are herein incorporated by reference.

Non-aqueous tackifying agents suitable for use in the present invention may be either used such that they form non-hardening coating or they may be combined with a multifunctional material capable of reacting with the non-aqueous tackifying agent to form a hardened coating. A “hardened coating” as used herein means that the reaction of the tackifying compound with the multifunctional material will result in a substantially non-flowable reaction product that exhibits a higher compressive strength in consolidated agglomerate than the tackifying compound alone with the particulates. In this instance, the non-aqueous tackifying agent may function similarly to a hardenable resin. Multifunctional materials suitable for use in the present invention include, but are not limited to, aldehydes such as formaldehyde, dialdehydes such as glutaraldehyde, hemiacetals or aldehyde releasing compounds, diacid halides, dihalides such as dichlorides and dibromides, polyacid anhydrides such as citric acid, epoxides, furfuraldehyde, glutaraldehyde or aldehyde condensates and the like, and combinations thereof. In some embodiments of the present invention, the multifunctional material may be mixed with the tackifying compound in an amount of from about 0.01 to about 50 percent by weight of the tackifying compound to effect formation of the reaction product. In some preferable embodiments, the compound is present in an amount of from about 0.5 to about 1 percent by weight of the tackifying compound. Suitable multifunctional materials are described in U.S. Pat. No. 5,839,510 issued to Weaver, et al., the relevant disclosure of which is herein incorporated by reference.

Solvents suitable for use with the non-aqueous tackifying agents suitable for use in the present invention include any solvent that is compatible with the non-aqueous tackifying agent and achieves the desired viscosity effect. The solvents that can be used in the present invention preferably include those having high flash points (most preferably above about 125° F.). Examples of solvents suitable for use in the present invention include, but are not limited to, butylglycidyl ether, dipropylene glycol methyl ether, butyl bottom alcohol, dipropylene glycol dimethyl ether, diethyleneglycol methyl ether, ethyleneglycol butyl ether, methanol, butyl alcohol, isopropyl alcohol, diethyleneglycol butyl ether, propylene carbonate, d'limonene, 2-butoxy ethanol, butyl acetate, furfuryl acetate, butyl lactate, dimethyl sulfoxide, dimethyl formamide, fatty acid methyl esters, and combinations thereof. It is within the ability of one skilled in the art, with the benefit of this disclosure, to determine whether a solvent is needed to achieve a viscosity suitable to the subterranean conditions and, if so, how much.

Aqueous tackifier agents suitable for use in the present invention are not significantly tacky when placed onto a particulate, but are capable of being “activated” (that is destabilized, coalesced and/or reacted) to transform the compound into a sticky, tackifying compound at a desirable time. Such activation may occur before, during, or after the aqueous tackifier agent is placed in the subterranean formation. In some embodiments, a pretreatment may be first contacted with the surface of a particulate to prepare it to be coated with an aqueous tackifier agent. Suitable aqueous tackifying agents are generally charged polymers that comprise compounds that, when in an aqueous solvent or solution, will form a non-hardening coating (by itself or with an activator) and, when placed on a particulate, will increase the continuous critical resuspension velocity of the particulate when contacted by a stream of water. The aqueous tackifier agent may enhance the grain-to-grain contact between the individual particulates within the formation (be they proppant particulates, formation fines, or other particulates), helping bring about the consolidation of the particulates into a cohesive, flexible, and permeable mass.

Suitable aqueous tackifying agents include any polymer that can bind, coagulate, or flocculate a particulate. Also, polymers that function as pressure sensitive adhesives may be suitable. Examples of aqueous tackifying agents suitable for use in the present invention include, but are not limited to: acrylic acid polymers; acrylic acid ester polymers; acrylic acid derivative polymers; acrylic acid homopolymers; acrylic acid ester homopolymers (such as poly(methyl acrylate), poly(butyl acrylate), and poly(2-ethylhexyl acrylate)); acrylic acid ester co-polymers; methacrylic acid derivative polymers; methacrylic acid homopolymers; methacrylic acid ester homopolymers (such as poly(methyl methacrylate), poly(butyl methacrylate), and poly(2-ethylhexyl methacrylate)); acrylamido-methyl-propane sulfonate polymers; acrylamido-methyl-propane sulfonate derivative polymers; acrylamido-methyl-propane sulfonate co-polymers; and acrylic acid/acrylamido-methyl-propane sulfonate co-polymers, derivatives thereof, and combinations thereof. The term “derivative” as used herein refers to any compound that is made from one of the listed compounds, for example, by replacing one atom in the base compound with another atom or group of atoms. Methods of determining suitable aqueous tackifying agents and additional disclosure on aqueous tackifying agents can be found in U.S. patent application Ser. No. 10/864,061 and filed Jun. 9, 2004 and U.S. patent application Ser. No. 10/864,618 and filed Jun. 9, 2004 the relevant disclosures of which are hereby incorporated by reference.

Some suitable tackifying agents are described in U.S. Pat. No. 5,249,627 by Harms, et al., the relevant disclosure of which is incorporated by reference. Harms discloses aqueous tackifying agents that comprise at least one member selected from the group consisting of benzyl coco di-(hydroxyethyl) quaternary amine, p-T-amyl-phenol condensed with formaldehyde, and a copolymer comprising from about 80% to about 100% C₁₋₃₀ alkylmethacrylate monomers and from about 0% to about 20% hydrophilic monomers. In some embodiments, the aqueous tackifying agent may comprise a copolymer that comprises from about 90% to about 99.5% 2-ethylhexylacrylate and from about 0.5% to about 10% acrylic acid. Suitable hydrophilic monomers may be any monomer that will provide polar oxygen-containing or nitrogen-containing groups. Suitable hydrophilic monomers include dialkyl amino alkyl(meth)acrylates and their quaternary addition and acid salts, acrylamide, N-(dialkyl amino alkyl)acrylamide, methacrylamides and their quaternary addition and acid salts, hydroxy alkyl(meth)acrylates, unsaturated carboxylic acids such as methacrylic acid or preferably acrylic acid, hydroxyethyl acrylate, acrylamide, and the like. These copolymers can be made by any suitable emulsion polymerization technique. Methods of producing these copolymers are disclosed, for example, in U.S. Pat. No. 4,670,501, the relevant disclosure of which is incorporated herein by reference.

Silyl-modified polyamide compounds suitable for use as an adhesive substance in the methods suitable for use in the present invention may be described as substantially self-hardening compositions that are capable of at least partially adhering to particulates in the unhardened state, and that are further capable of self-hardening themselves to a substantially non-tacky state to which individual particulates such as formation fines will not adhere to, for example, in formation or proppant pack pore throats. Such silyl-modified polyamides may be based, for example, on the reaction product of a silating compound with a polyamide or a mixture of polyamides. The polyamide or mixture of polyamides may be one or more polyamide intermediate compounds obtained, for example, from the reaction of a polyacid (e.g., diacid or higher) with a polyamine (e.g., diamine or higher) to form a polyamide polymer with the elimination of water. Other suitable silyl-modified polyamides and methods of making such compounds are described in U.S. Pat. No. 6,439,309 issued to Matherly, et al., the relevant disclosure of which is herein incorporated by reference.

In one embodiment, the present invention provides a method comprising: providing a treatment fluid that comprises an aqueous base fluid, optionally a non-aqueous tackifying agent, and a degradable gemini surfactant composition of the present invention; and introducing the treatment fluid into a subterranean formation.

The degradable gemini surfactant compositions of the present invention may be used in an emulsified treatment fluid (e.g., emulsified acids for stimulation treatments, emulsified fracturing fluids, emulsified drilling fluids, etc.). As referred to herein, the term “emulsified treatment fluid” refers to any emulsified fluid that has a continuous phase and a discontinuous phase. These include water-in-oil (“w/o”) emulsions as well as oil-in-water (“o/w”) emulsions. In an o/w emulsion, the aqueous phase is the continuous (or external) phase and the oleaginous phase is the discontinuous (or internal) phase. In a w/o emulsion (or invert emulsion), the aqueous phase is the discontinuous phase and the oleaginous phase is the continuous phase. The emulsified treatment fluids suitable for use in the present invention may be used in any suitable subterranean application where an emulsion may be used, including, but not limited to, drilling operations (e.g., as a drilling fluid or a drill-in fluid), fracturing treatment (e.g., as a fracturing fluid), well bore cleanups, viscous sweeps, and sand control treatments (e.g., as a gravel-packing fluid).

In some embodiments, the present invention provides an emulsified treatment fluid comprising an oleaginous phase, an aqueous phase, and an emulsifying agent that comprises a degradable gemini surfactant of the present invention.

Generally, the emulsified treatment fluids suitable for use in the present invention are suitable for use in a variety of subterranean applications where an o/w emulsion or a w/o emulsion is suitable, e.g., emulsified acids for stimulation treatments, emulsified fracturing fluids, emulsified drilling fluids, etc. For example, the emulsified treatment fluids may be useful for facilitating the formation of w/o emulsions that may be used in a variety of subterranean operations including, drilling operations (e.g., as a drilling fluid or a drill-in fluid), fracturing treatment (e.g., as a fracturing fluid), well bore cleanups, viscous sweeps, and sand control treatments (e.g., as a gravel-packing fluid), acidizing treatments, stimulation treatments, consolidation treatments, fines control treatments, and cementing operations. A drill-in fluid is a drilling fluid formulated for drilling the reservoir portion of the subterranean formation.

The oleaginous phase of the emulsified treatment fluids may comprise any oleaginous fluid suitable for use in emulsions used in subterranean applications. The oleaginous fluid may be from natural or synthetic sources. Examples of suitable oleaginous fluids include, but are not limited to, α-olefins, internal olefins, alkanes, aromatic solvents, cycloalkanes, liquefied petroleum gas, kerosene, diesel oils, crude oils, heavy oils, gas oils, fuel oils, paraffin oils, mineral oils, low toxicity mineral oils, olefins, esters, amides, synthetic oils (e.g., polyolefins), polydiorganosiloxanes, siloxanes, organosiloxanes, ethers, acetals, dialkylcarbonates, hydrocarbons, and combinations thereof.

The amount of the oleaginous phase to include in the emulsified treatment fluid depends on a number of factors, including the particular degradable gemini surfactant used, the type of emulsion (e.g., o/w or w/o), the desired application, and rheology. For example, in certain embodiments, such as a stimulation embodiment, the emulsified treatment fluid should have sufficient viscosity for proppant transport. In some embodiments, for an o/w emulsion, the oleaginous phase may be present in an amount in the range of from about 10% to about 65% by volume of the emulsified treatment fluid. In some embodiments, for a w/o emulsion, the oleaginous phase may be present in an amount in the range of from about 20% to about 90% by volume of the emulsified treatment fluid.

For the emulsion embodiments, the emulsified treatment fluids may also comprise an aqueous phase. Generally, the aqueous phase may comprise an aqueous liquid. Suitable aqueous liquids may include, but are not limited to, freshwater, seawater, saltwater, brines (e.g., natural or formulated brines), and combinations thereof. The aqueous liquid may be from any source, provided that it does not contain an excess of compounds that may adversely affect the emulsified treatment fluid.

The amount of the aqueous phase to include in the emulsified treatment fluid depends on a number of factors, including the particular surfactant used, the type of emulsion (e.g., o/w or w/o), the desired application, and rheology. In some embodiments, for an o/w emulsion, the aqueous phase may be present in an amount in the range of from about 35% to about 90% by volume of the emulsified treatment fluid. In some embodiments, for a w/o emulsion, the aqueous phase generally may be present in an amount in the range of from about 10% to about by 80% volume of the emulsified treatment fluid.

Depending on the particular application, the emulsified treatment fluids suitable for use in the present invention may further comprise any of a variety of additional additives. Examples of suitable additives include, but are not limited to, proppant particulates, gravel particulates, weighting agents, organophilic clays, bridging agents, fluid loss control agents, wetting agents, corrosion inhibitors, scale inhibitors, fluid loss control additives, gas, paraffin inhibitors, asphaltene inhibitors, catalysts, hydrate inhibitors, iron control agents, clay control agents, biocides, friction reducers, combinations thereof, and the like. The particular additives included in the treatment fluids should not adversely affect other components of the emulsified treatment fluid. Individuals skilled in the art, with the benefit of this disclosure, will recognize the types of additives to include for a particular application.

An example method suitable for use in the present invention comprises: providing an emulsified treatment fluids suitable for use in the present invention that comprises a degradable gemini surfactant composition of the present invention, an oleaginous phase, and an aqueous phase having a pH of about 3 to about 12; and introducing the emulsified treatment fluid into a subterranean formation. In certain acidizing embodiments, an example method suitable for used in the present invention comprises: a degradable gemini surfactant composition of the present invention, an oleaginous phase, and an aqueous phase having a pH in the range of about 0 to about 4; and introducing the emulsified treatment fluid into a subterranean formation. Introducing the emulsified treatment fluid into the subterranean formation includes introducing the emulsified treatment fluid into a well bore that penetrates the subterranean formation. As previously discussed, the emulsified treatment fluid may be an o/w or a w/o emulsion.

In the drilling embodiments, the emulsified treatment fluids may be used in drilling at least a portion of a well bore that penetrates the subterranean formation. For example, the emulsified treatment fluids may be used as a drilling fluid or a drill-in fluid.

In another embodiment, the emulsified treatment fluids suitable for use in the present invention may be used in a sand control treatment (e.g., as a gravel-packing fluid). In the sand control embodiments, the emulsified treatment fluids may further comprise gravel particulates, wherein at least a portion of the gravel particulates may be deposited within or adjacent to a portion of the subterranean formation to form a gravel pack. In the fracturing embodiments, the emulsified treatment fluid may be introduced into the subterranean formation at or above pressure sufficient to create or enhance one or more fractures in the subterranean formation.

In another embodiment, the emulsified treatment fluid may be used in a fracturing application wherein a fracturing fluid comprising a degradable gemini surfactant composition of the present invention is introduced into a well bore at a pressure sufficient to create or enhance a fracture therein.

Another example of a method suitable for use in the present invention comprises utilizing the emulsified treatment fluids suitable for use in the present invention to facilitate the flow of the oleaginous phase through a conduit, for example, to facilitate the flow of heavy oil through a pipeline. An example of such a method may comprise: providing an emulsified treatment fluid suitable for use in the present invention that comprises an orthoester-based surfactant, an oleaginous phase, and an aqueous phase having a pH in the range of about 3 to about 12; flowing the emulsified treatment fluid through a conduit; reducing the pH of the aqueous phase so as to facilitate degradation of at least a portion of the orthoester-based surfactant, thereby facilitating the separation of the oleaginous phase and the aqueous phase. Since the emulsified treatment fluid may be broken by degradation of the orthoester-based surfactant, when the pH of the aqueous phase is reduced, the oleaginous phase and the aqueous phase should separate. Among other things, this may allow for recovery of the oleaginous fluid at a desired location. In most embodiments, the pH of the aqueous phase may reduced at the receiving end of the pipeline where desired to recover the oleaginous fluid. In certain embodiments, the oleaginous phase may be a heavy oil. Where a heavy oil is used, the emulsified treatment fluid may be used to facilitate the flow of the heavy through a conduit, such as a pipeline.

In some embodiments, the present invention provides a foamed treatment fluid suitable for use in the present invention made comprising a base fluid, a gas, and a foaming agent that comprises a degradable gemini surfactant.

Foamed treatment fluids may be used in a variety of subterranean treatments, such as drilling operations, well bore cleanup operations, hydraulic fracturing, fracture acidizing, sand control treatments, and the like. A foamed treatment fluid may be prepared by mixing an aqueous fluid containing a foam stabilizing surfactant mixture with a gas (such as air, nitrogen, carbon dioxide, or combinations thereof). Generally, the foam stabilizing surfactant mixture facilitates the foaming of the aqueous fluid and also may stabilize the resultant foamed fluid formed therewith. Foamed treatment fluids may effectively carry particulates and also may require a smaller amount of gelling agent, reducing the amount of residue left in the subterranean formation by the gelling agent. Additionally, foamed treatment fluids have low fluid loss properties, potentially reducing or removing the need for a fluid loss control additive. Furthermore, foaming a treatment fluid generally reduces the water requirement, thereby minimizing problems associated with clay swelling.

The foamed treatment fluid suitable for use in the present invention may comprise a base fluid, gas, and a foaming agent that comprises a degradable gemini surfactant of the present invention.

As used herein, the term “base fluid” is any aqueous or non-aqueous liquid fluid that may be, among other things, fresh water, salt water, a brine, seawater, a mineral oil, a synthetic oil, an ester, and combinations thereof. The base fluid may be from any source, provided that it does not prohibit the use of the foamed treatment fluids of the present invention.

The gas included in the treatment fluids suitable for use in the present invention may be any gas suitable for foaming a treatment fluid, including, but not limited to, nitrogen, carbon dioxide, and air, and derivatives thereof and combinations thereof. Generally, the gas should be present in the foamed treatment fluids suitable for use in the present invention in an amount sufficient to form a foam. As used herein, “foaming”and “foamed” also encompass“commingling” and “commingled” fluids.

In certain embodiments, the gas may be present in the foamed treatment fluids suitable for use in the present invention in an amount in the range of from about 5% to about 98% by volume of the foamed treatment fluid, exclusive of the volume of the gas. In certain embodiments, the gas may be present in the foamed treatment fluids suitable for use in the present invention in an amount in the range of from about 30% to about 98% by volume of the foamed treatment fluid, exclusive of the volume of the gas. In certain embodiments, the gas may be present in the foamed treatment fluids suitable for use in the present invention in an amount in the range of from about 55% to about 98% by volume of the foamed treatment fluid, exclusive of the volume of the gas.

The degradable gemini surfactant composition, in these embodiments, should be present in a treatment fluid in an amount sufficient to impart the desired stability and viscosity (e.g., sufficient viscosity to divert flow, reduce fluid loss, suspend particulates, etc.) to the treatment fluid. In certain embodiments, the degradable gemini surfactant composition may be present in the treatment fluid in an amount in the range of from about 0.1% to about 20% by weight of the fluid. In certain embodiments, the degradable gemini surfactant composition may be present in an amount in the range of from about 1% to about 10% by weight of the fluid. In certain embodiments, the degradable gemini surfactant composition may be present in an amount of about 7% by weight of the fluid.

As will be recognized by those of ordinary skill in the art, with the benefit of this disclosure, a wide variety of additional additives may be included in the foamed treatment fluids of the present invention. Examples of suitable additives include, but are not limited to, gelling agents, gel breakers, proppant particulates, gravel particulates, defoaming agents, clay stabilizers, scale inhibitors, fluid loss control additives, and combinations thereof.

The foamed treatment fluids suitable for use in the present invention may be used in any suitable subterranean treatment where a foamed treatment fluid emulsion may be used, including, but not limited to, drilling operations (e.g., as a drilling fluid or a drill-in fluid), fracturing treatment (e.g., as a fracturing fluid), well bore cleanups, and sand control treatments (e.g., as a gravel packing fluid). An example method suitable for use in the present invention of treating a subterranean formation comprises: providing a foamed treatment fluid suitable for use in the present invention that comprises an aqueous fluid, a gas, and a foaming agent that comprises a degradable gemini surfactant composition of the present invention; and introducing the foamed treatment fluid into a well bore that penetrates the subterranean formation.

The treatment fluids of the present invention and/or any component thereof may be prepared at a job site, or they may be prepared at a plant or facility prior to use, and may be stored for some period of time prior to use. In certain embodiments, the preparation of these synergistic viscosification additives and treatment fluids of the present invention may be done at the job site in a method characterized as being performed “on the fly.” The term “on-the-fly” is used herein to include methods of combining two or more components wherein a flowing stream of one element is continuously introduced into flowing stream of another component so that the streams are combined and mixed while continuing to flow as a single stream as part of the on-going treatment. Such mixing can also be described as “real-time” mixing.

The methods and treatment fluids of the present invention may be used during or in preparation for any subterranean operation wherein a fluid may be used. Suitable subterranean operations may include, but are not limited to, drilling operations, hydraulic fracturing treatments, sand control treatments (e.g., gravel packing), acidizing treatments (e.g., matrix acidizing or fracture acidizing), “frac-pack” treatments, well bore clean-out treatments, and other suitable operations where a treatment fluid of the present invention may be useful. In one embodiment, the present invention provides a method that comprises: providing a treatment fluid that comprises an aqueous base fluid, a non-aqueous tackifying agent, and a degradable gemini surfactant; and introducing the treatment fluid into a subterranean formation. In certain embodiments, the treatment fluid may be introduced into the subterranean formation at or above a pressures sufficient to create or enhance at least one fracture in the subterranean formation. In certain embodiments, the treatment fluid may comprise a plurality of gravel particulates, and the methods may further comprise depositing at least a portion of those particulates in a desired area in a well bore, e.g., to form a gravel pack, provide some degree of sand control in the subterranean formation, and/or prevent the flow of particulates from an unconsolidated portion of the subterranean formation (e.g., a propped fracture) into a well bore.

To facilitate a better understanding of the present invention, the following examples of certain aspects of some embodiments are given. In no way should the following examples be read to limit, or define, the entire scope of the invention.

### EXAMPLE

General procedure for the synthesis of alkylester chloride 2: Synthesis of 2b is typical. To a 500 mL round bottom flask equipped with a magnetic stirrer and an inert atmosphere, suspended 1-hexadecanol (100 g, 0.412 mol) in 200 mL of dichloromethane. To this solution was added chloroacetyl chloride (46.58 g, 0.412 mol) and the resulting suspension was stirred at room temperature. The suspended 1-hexadecanol slowly dissolved over 15 min and afforded a clear transparent solution. The solution was additionally stirred for 8 h at room temperature and finally it was refluxed for 30 min. The solution was cooled and the dichloromethane was removed under reduced pressure using rotavapor to afford the final product (2b) in quantitative yield. The material was used in the subsequent reactions without further purification.

General procedure for the synthesis of quaternary ammonium surfactant 6, 8 and 10: Synthesis of 6b is typical. To a 1 L round bottom flask equipped with a magnetic stirrer and an inert atmosphere, cetylester chloride 2b (25 g, 0.072 mol) and 250 mL of acetone are added. To this solution, N,N,N′,N″,N″-pentamethyldiethylenetriamine 5 (6.25 g, 0.036 mol) is added, and the solution is stirred for 72 h at room temperature. The quaternary ammonium surfactant formed by the reaction precipitates out of the acetone solution. The precipitates formed are filtered by Buckner funnel and washed with cold acetone. The white precipitates obtained are dried under vacuum to afford 28 g (yield 89.6%) of the product 6b.

General procedure for the preparation of viscosified fluid: To 100 ml of water in a Waring blender the surfactant is added (see Table 1) and the resulting mixture is stirred at 2000-2500 rpm for 3-5 minutes. Appropriate amount of potassium chloride salt and/or sodium salicylate is added to the mixture and stirred additionally for 3 minutes. The material is then centrifuged to separate the air bubbles out of the viscous fluid before any rheological measurements are performed on the samples.

General procedure for the rheological measurements and techniques: Equipment: All rheological measurements were performed on the “STRESSTECH-HR” rotational stress-controlled rheometer (Rheologica, Sweden) equipped with a seal cell system. It is manufactured and marketed by ATS RheoSystems, Bordentown, N.J., USA. The seal cell has been designed for direct rheological measurements of materials at a maximum pressure of 60 psi. The measuring system has a rotating bob-in-cup fixture, that is used in this description. In this work, a moony bob with bob section length of 45 mm, radius 12.5 mm and cup with a radius of 13.75 mm was employed yielding the radius ratio (bob/cup) of 0.9. Based on the radius ratio, the measuring system can be classified as a narrow gap Couette system, so that the momentum diffusion (shear rate) across the gap width is assumed to be in a linear relationship with the rotational speed of the bob. The Couette system is constructed from Stainless 316 grade, which can withstand relatively high temperatures of the experiment and does not react with the sample. During rheological measurements, the bob was rotated at a constant angular speed while the cup remained stationary.

To ensure that each test sample encounters the same initial thermal and structural conditions (which is important for our surfactant samples), the following treatments are performed to condition the samples prior to rheological measurements. First, the samples are loaded into the cup prior to pressurizing the system with a nitrogen gas pressure of 30 psi that is sufficient enough to prevent the aqueous base fluid from boiling. Then, the measuring system with the positioned sample is heated at the desired temperature with the heating rate of 2° C./min. During the heating process each sample is sheared at a fixed shear rate of 300 l/s, while shear stress and hence apparent viscosity are monitored as a function of time. After the desired temperature is achieved, each sample is held at the temperature for an additional 10 minutes to ensure that the targeted temperature is attained and homogenized. Then, rheological measurements are performed using step down in shear rate technique at which shear rate is decreasing in a logarithmic fashion from 300 to 0.03 s⁻¹. At a given shear rate value, each sample is sheared for 60 seconds. The torque value and hence shear stress is calculated by integrating the torque values recorded over the last ten seconds of experiment. This is done to ensure that the effect of stress overshoot is not incorporated into our measuring data.

Rheological measurements at other temperatures was performed by repeating the second and third steps above. In this study, the rheological measurements of gels are carried out at four different temperatures of 23° C., 30° C., 40° C. and 50° C. The effects of cooling on rheological behavior of our surfactant gels were investigated by cooling the heated sample down to 40° C., 30° C. and 23° C., respectively. The cooling process was similar to that of the heating step. At the end of each steady shear test, the sample was removed and discharged.

Synthesis of gelling surfactants: The degradable quaternary ammonium salt surfactants may be prepared as shown in Scheme 1-2. The hydrolyzable ester group between the hydrophobic tail and the quaternary ammonium hydrophilic head group is introduced by first reacting n-alkyl alcohol with chloroacetyl chloride followed by the reaction of the alkylester chloride formed, with tertiary amine.

From the data, it is believed that the surfactants shown as prepared in Scheme 1 below are capable of gelling an aqueous based fluid, but higher concentrations of the surfactant are needed to get appreciable viscosity.

To illustrate a synthesis of a gemini surfactant, alkylester chloride 2a-c was reacted with N,N,N′,N″,N″-pentamethyldiethylenetriamine 5 and bis[2-(N,N-dimethylamine)ethyl]ether 7 in acetone, at room temperature, for 72 hours to afford gemini surfactants 6a-c and 8a-c respectively in quantitative yield (see Scheme 1 below for synthesis description).

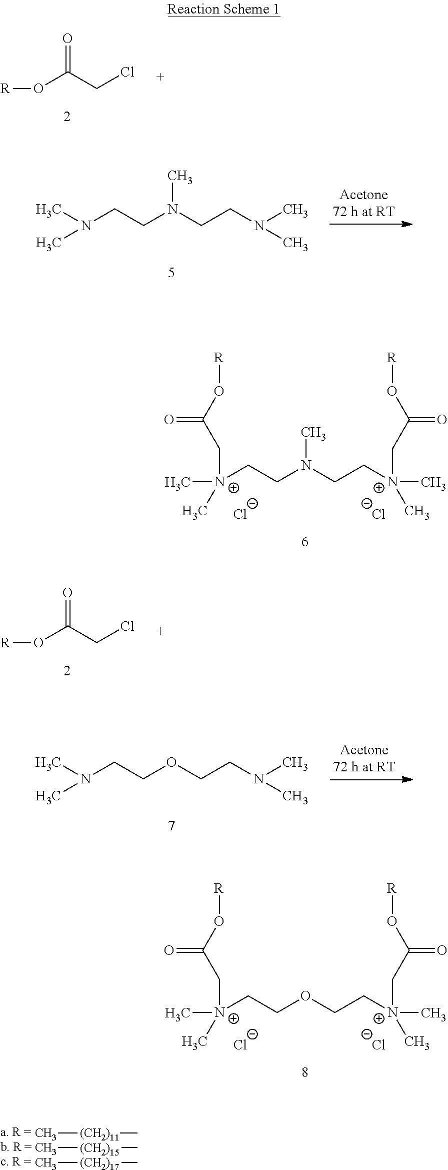

As shown in Reaction Scheme 2, gemini surfactants with a spacer length of 2 are synthesized to illustrate possible difference in the gelling properties in comparison to the gelling properties of gemini surfactant with a spacer length of 5 (as shown in Scheme 1).

Alkylester chloride (2a,b; see R description for a and b in the illustration) is reacted with N,N,N′,N′-tetramethylethylenediamine 9 in acetone, at room temperature, for 72 h to afford Gemini surfactant 10a,b in quantitative yield (Scheme 2). The Gemini surfactant 10a,b contains a spacer length of 2 whereas surfactant 6a-c and 8a-c contains a spacer length of 5.

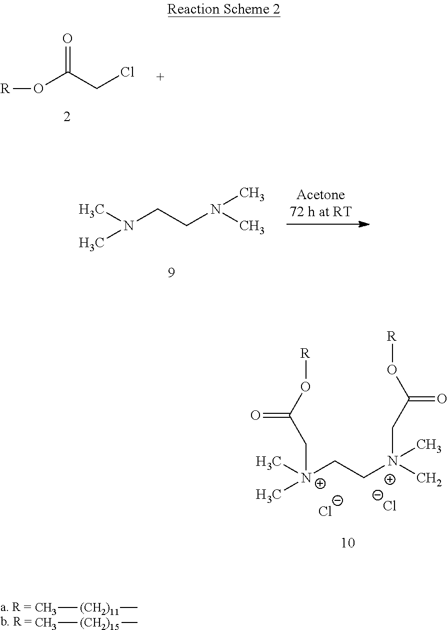

Gelling and rheological properties of degradable VES: The surfactants prepared according to reaction schemes 1-2 above are used as thickening agents in aqueous based systems to illustrate gelling properties. Table 1 provides another way of looking at the collected data from the experiments performed with the concentration of surfactant, KCl salt and sodium salicylate in the gelled fluid. The rheological properties of the system are measured at 4 different temperatures, i.e., 23, 30, 40 and 50° C. The water was used to prepare the gels, with the final sample pH not adjusted.

TABLE 1

| | | | | Sodium | Comments Based |
|---|---|---|---|---|---|
| Sample | | Surfactant | KCl Salt | salicylate | on Visual |
| Number | Surfactant | (%) | (%) | (%) | Observation |
| 1 | 6b | 2.0 | — | 0.36 | Viscous fluid |
| 2 | 6b | 2.0 | 2.0 | — | Viscous fluid |
| 3 | 6c | 1.0 | 2.0 | — | Viscous fluid |
| 4 | 6c | 1.5 | 1.5 | 0.1 | Milky fluid and |
| | | | | | gel with increase |
| | | | | | of temperature; |
| | | | | | Precipitated |
| | | | | | on standing |
| 5 | 10b | 1.5 | — | 0.6 | Viscous fluid |
| 6 | 10b | 1.5 | 2.0 | Added | Viscous fluid |
| | | | | sodium | |
| | | | | xylene | |
| | | | | sulfonate | |
| | | | | 0.4 | |
| 7 | 10b | 3.0 | — | 0.6 | Viscous fluid |
| 8 | 10b | 3.0 | — | 1.2 | Viscous fluid |
| 9 | 10b | 3.0 | 2.0 | 0.3 | Viscous fluid |

Surfactant 6c and 10c with a bigger alkyl group (C₁₈) appear to be less soluble in water due to the higher Kraft Temperature, which is the temperature above which a mixture of solid surfactant and water turns into solution. The increase in the alkyl chain is believed to increase the Kraft Temperature. Therefore, due to the lower solubility of these surfactants, they do form viscous fluids at room temperature. As the temperature of mixture is increased above the Kraft Temperature the surfactants appear to viscosify water. Also, on standing, the fluid at room temperature after dissolution at high temperature the surfactant precipitates out of the solution slowly. Replacing the chloride counterion of surfactant with a bigger counterion like sodium salicylate increases the Kraft Temperature of the surfactant which again leads to precipitation of surfactant from the solution. Therefore, it appears that surfactants with bigger alkyl group are not as suitable for gelling water at room temperature (Table 1, sample 4), but can be useful in application where relation at higher temperature is needed. On the other hand, surfactants with alkyl chain of C₁₂ and C₁₆ (6a,b and 10a,b) are suitable to gel the water or aqueous fluid at room temperature. The gel formation can be assisted by addition of the counterion (e.g., sodium salicylate, sodium xylene sulfonate) or by addition of salt to the solution of surfactant in aqueous solution.

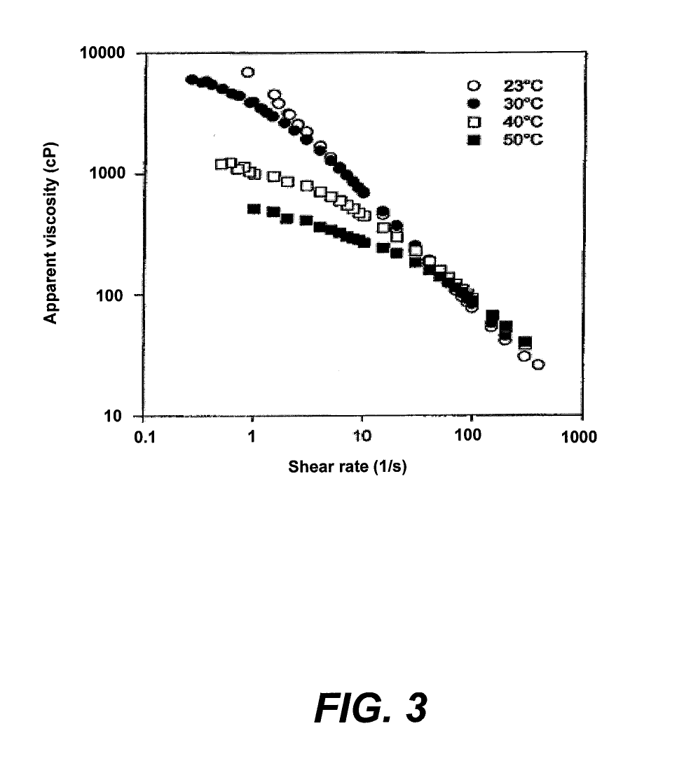

FIG. 3 shows typical flow behavior of fluid obtained from the sample 5 (Table 1) in the form of apparent viscosity versus shear rate plots. Over a temperature range of 23-50° C., the observed rheological behavior of fluid is typical of shear thinning characteristics at which the apparent viscosity of the surfactant at any given temperature is not constant, but appears to decrease dramatically with increasing shear rate. At any given shear rate, the surfactant viscosity decreases with increasing temperature.

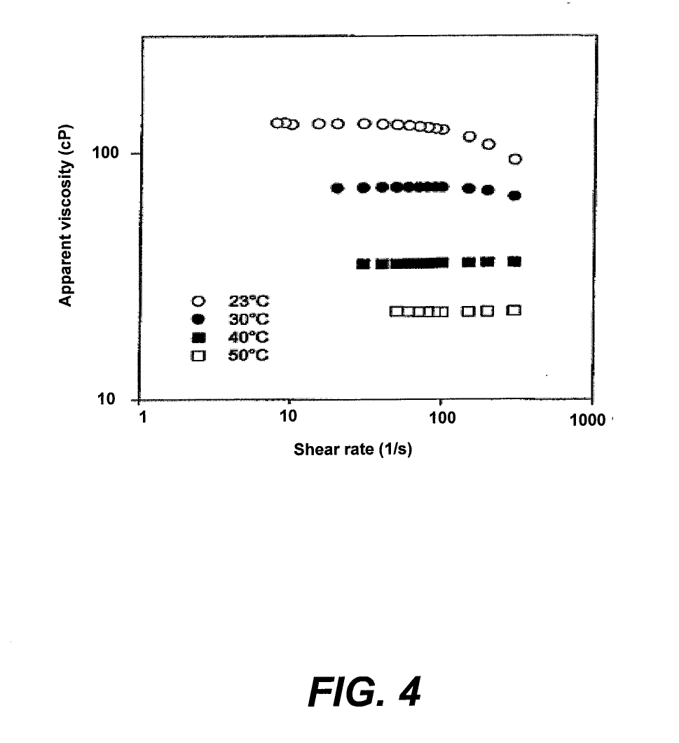

The rheological properties of sample 8 (containing 3 wt % of surfactant 10b and 1.2 wt % of sodium salicylate in water) were measured under a rich nitrogen gas conditions at a temperature from 23-50° C. The results are shown in FIG. 4. FIG. 4 shows that sample 8 exhibits Newtonian characteristics at 5.0≦γ≦100 s⁻¹ for all temperatures ranges tested. At γ≦100 s⁻¹, slight shear thinning behavior is observed especially with the sample tested at 23° C.

It is apparent that the amount of surfactant and counter ion is causing some structural variation in the surfactant micelles leading to the apparent Newtonian behavior. A comparison of this result with sample 7 with half the amount of surfactant 10b and half the amount of counterion reveal that the sample becomes less viscous and has less shear rate sensitivity when the concentration of either the counter ion or surfactant is increased. This could be caused by the difference in their chemical compositions and has a direct effect on the formation of micellization structure of the surfactant.

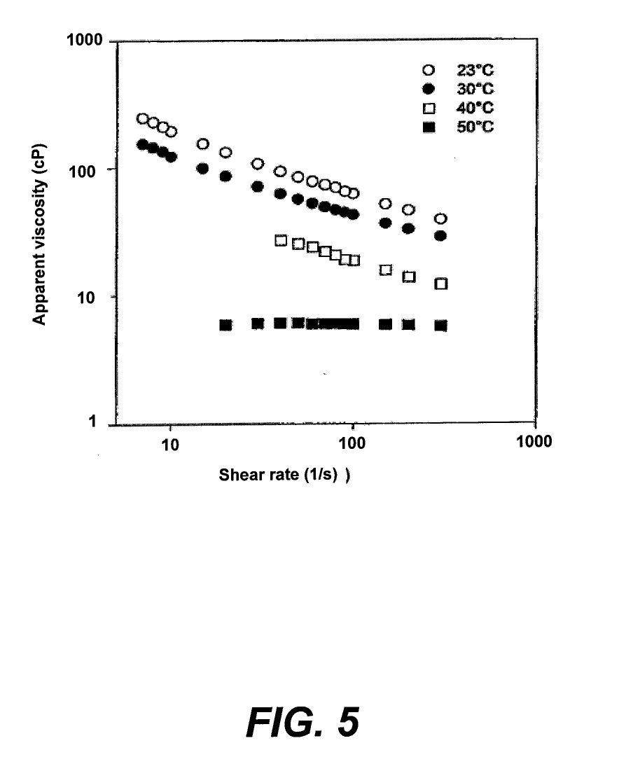

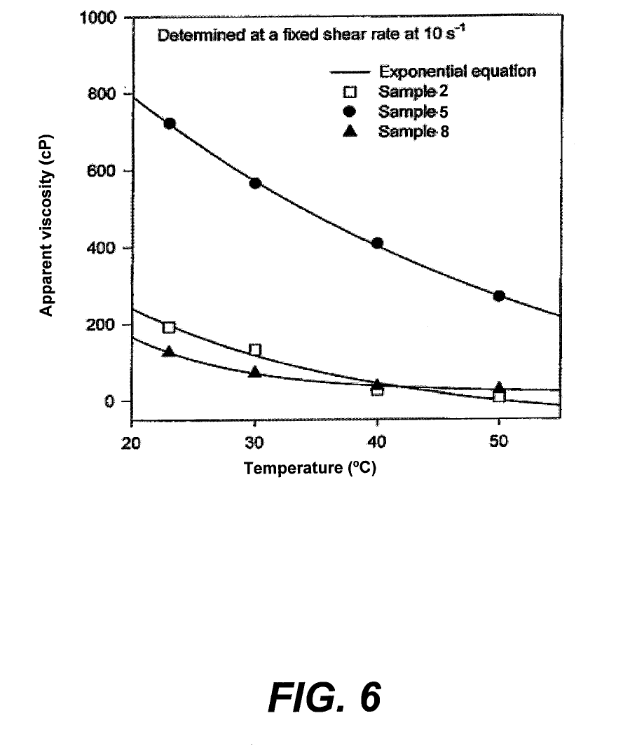

FIG. 6 shows the flow behavior of samples 2, 5, and 8. The results demonstrate shear thinning characteristics of the material over the temperature range tested. FIG. 5 shows the flow behavior of sample 2, which contains 2.0 wt % of surfactant 6b and 2 wt % of KCl in water. The results demonstrate shear thinning characteristics of the material over the temperature range tested. A comparison of the viscosity values between samples 5 and 2 has found that sample 5 is more viscous than sample 2 over the temperature range tested. This is in agreement with the reported literature data which demonstrated that the Gemini surfactants fluids/gels with a spacer length of 2 are more viscous than the fluids/gels prepared by surfactant with a spacer length of 5. Viscosity values of the surfactant samples tested at 23° C. at shear rate of 10, 50 and 100 s⁻¹ are given in Table 2.

TABLE 2

| Sample | 10 s⁻¹ | 50 ⁻¹ | 100 ⁻¹ |
|---|---|---|---|
| Number | (cP) | (cP) | (cP) |
| 1 | 49 | 35 | 31 |
| 2 | 192 | 80 | 61 |
| 3 | 26 | 24 | 20 |
| 4 | 224 | 51 | 33 |
| 5 | 670 | 144 | 76 |
| 6 | 55 | 46 | 42 |
| 7 | 603 | 144 | 78 |
| 8 | 129 | 128 | 123 |
| 9 | 534 | 174 | 99 |

Effects of temperature: The rheological property data in FIGS. 3-6 indicate that the surfactant samples showed a decrease in the apparent viscosity with increasing temperature. FIG. 6 shows, the apparent viscosity of samples 2, 5 and 8 determined at a constant shear rate of 10 s⁻¹ plotted directly as a function of temperature. It is apparent from the plotted data that sample 8 is less sensitive to temperature in comparison to other samples prepared.

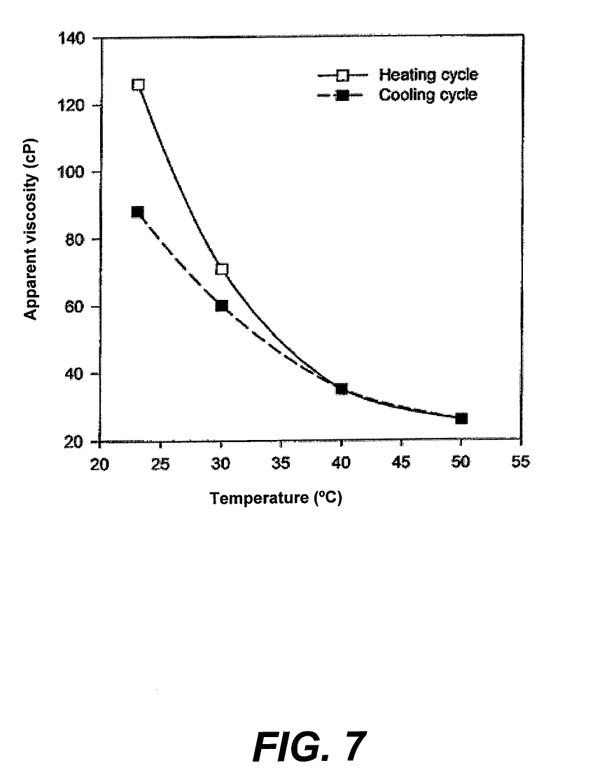

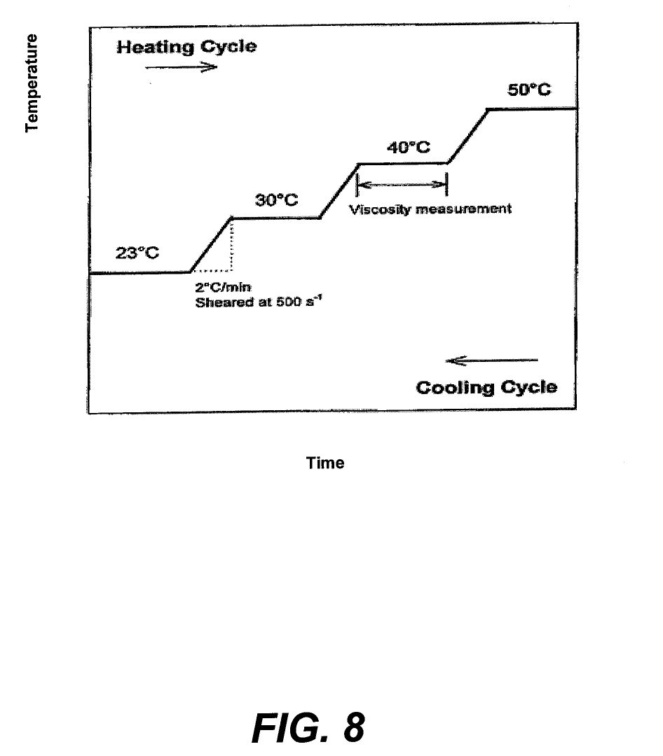

The experimental results can be satisfactorily fitted by an exponential equation, η=A exp(−B/T), where η is the apparent viscosity at a fixed shear rate, T is the temperature, A is the interception on viscosity axis and B is the slope of the curve. On comparing parameter B values of samples 2, 5 and 8 it has been found that sample 2 has the lower value of the B parameter at 0.026, whereas the sample 8 has the highest value at 0.106. The different in values of the B parameter mean that the viscosity of sample 2 is the most sensitive to temperature and vice versa. Value of the parameters A and B for all the surfactant samples prepared are given in Table 3. Effects of temperature history on viscosity of surfactant samples are also studied in this work, and the results are given in FIG. 7. In addition, FIG. 8 shows the temperature variation for the surfactant gel samples tested herein. The results demonstrate viscosity hysteresis behavior of the sample 2 as the cooling-curve always lies below the heating-curve, indicating effects of temperature history on flow properties of the material. This might be due to the temperature history effect on the packing and arrangement of micellization structure of the surfactant.

TABLE 3

| Sample | | | | | | |
|---|---|---|---|---|---|---|
| Number | 23° C. | 30° C. | 40° C. | 50° C. | A (cP) | -B (cP/° C.) |
| 1 | 49 | 71.4 | 35 | 22 | 97.8 | 0.028 |
| 2 | 192 | 122 | 26 | 6 | 5012.2 | 0.136 |
| 3 | 26 | 18 | 11 | 3 | 178.26 | 0.077 |
| 4 | 224 | 135 | n/a | 13 | 2985.7 | 0.108 |
| 5 | 670 | 670 | 446 | 267 | 1717.4 | 0.035 |
| 6 | 55 | n/a | n/a | n/a | n/a | n/a |
| 7 | 603 | 575 | 407 | n/a | 1092.4 | 0.024 |
| 8 | 129 | 71 | 35 | 22 | 536.6 | 0.065 |
| 9 | 534 | 36.5 | 30 | 22 | 4980.7 | 0.116 |

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of or “consist of the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an”, as used in the claims, are defined herein to mean one or more than one of the element that it introduces. Also, the terms in the claims have their plain, ordinary meaning unless otherwise explicitly and clearly defined by the patentee. If there is any conflict in the usages of a word or term in this specification and one or more patent or other documents that may be incorporated herein by reference, the definitions that are consistent with this specification should be adopted.

1-41. (canceled)

42. A subterranean treatment fluid comprising a degradable gemini surfactant composition described by the following formula: 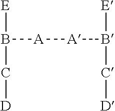
wherein:
A and A′ are spacers and may be a hydrophobic group or a hydrophilic group;
B is an ion and may contain a quaternary nitrogen, a sulfonate, or a phosphate; and,
A, A′, B, B′, C, C, E and E′ may be an alkyl, an aryl, a sugar, an ester, an ether, and any combination thereof;
D and D′ may be an aryl, a sugar, an ester, an ether, and any combination thereof; and,
A′, D′, and E′ are optional.

43. The degradable gemini surfactant of claim 42 wherein B comprises a cationic, anionic, nonionic, or zwitterionic moiety.

44. The subterranean treatment fluid of claim 42 wherein the degradable gemini surfactant is added in a range of about 0.1% to 20% by weight of the treatment fluid.

45. The subterranean treatment fluid of claim 42 wherein the treatment fluid comprises a base fluid selected from the group consisting of fresh water, salt water, a brine, seawater, a mineral oil, a synthetic oil, an ester, and combinations thereof.

46. The subterranean treatment fluid of claim 42 wherein the treatment fluid further comprises a resin selected from the group consisting of an organic resin, an epoxy based resin, a novolak resin, a polyepoxide resin, a phenol-aldehyde resin, an urea-aldehyde resin, a urethane resin, a phenolic resin, a furan resin, a furan/furfuryl alcohol resin, a phenolic/latex resin, a phenol formaldehyde resin, a polyester resin, a polyurethane resin, acrylate resins, a hybrid and a copolymer of any of these, and any combination thereof.

47. The subterranean treatment fluid of claim 42 wherein the treatment fluid further comprises additives selected from the group consisting of a salt, a soap, a co-surfactant, a carboxylic acid, an acid, a fluid loss control additive, a gas, a foamer, a corrosion inhibitor, a scale inhibitor, a catalyst, a clay control agent, a biocide, a friction reducer, an antifoam agent, a bridging agent, a dispersant, a flocculant, an H2S scavenger, a CO₂ scavenger, an oxygen scavenger, a lubricant, a viscosifier, a breaker, a weighting agent, a relative permeability modifier, a resin, a particulate material, a wetting agent, a coating enhancement agent, and any combination thereof.

48. The subterranean treatment fluid of claim 42 wherein the treatment fluid is foamed with a gas.

49. The foamed treatment fluid of claim 48 wherein the gas is added in the range of about 5% to about 98% by volume of the foamed treatment fluid.

50. A degradable gemini surfactant described by the following formula: 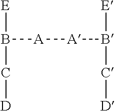
wherein:
A and A′ are spacers and may be a hydrophobic group or a hydrophilic group;
B is an ion and may contain a quaternary nitrogen, a sulfonate, or a phosphate; and,
A, A′, B, B′, C, C, E and E′ may be an alkyl, an aryl, a sugar, an ester, an ether, and any combination thereof;
D and D′ may be an aryl, a sugar, an ester, an ether, and any combination thereof; and,
A′, D′, and E′ are optional.

51. The degradable gemini surfactant of claim 50 wherein B comprises a cationic, anionic, nonionic, or zwitterionic moiety.

52. An emulsified treatment fluid comprising an oleaginous phase, an aqueous phase, and an emulsifying agent that comprises a degradable gemini surfactant described by the following formula: 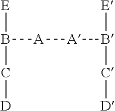
wherein:
A and A′ are spacers and may be a hydrophobic group or a hydrophilic group;
B is an ion and may contain a quaternary nitrogen, a sulfonate, or a phosphate; and,
A, A′, B, B′, C, C, E and E′ may be an alkyl, an aryl, a sugar, an ester, an ether, and any combination thereof; and,
D and D′ may be an aryl, a sugar, an ester, an ether, and any combination thereof.

53. The emulsified treatment fluid of claim 52 wherein the oleaginous phase is selected from a group consisting of an a-olefin, an internal olefin, an alkane, an aromatic solvent, a cycloalkane, a liquefied petroleum gas, kerosene, a diesel oil, a crude oil, a heavy oil, a gas oil, a fuel oil, a paraffin oil, a mineral oil, a low toxicity mineral oil, an olefin, an ester, an amide, a synthetic oil, a polydiorganosiloxane, a siloxane, an organosiloxane, an ether, an acetal, a dialkylcarbonate, a hydrocarbon, and any combination thereof.

54. The emulsified treatment fluid of claim 52 wherein the aqueous phase is selected from a group consisting of freshwater, seawater, saltwater, a brine, and any combination thereof.

55. The degradable gemini surfactant of claim 52 wherein B comprises a cationic, anionic, nonionic, or zwitterionic moiety.

56. The subterranean treatment fluid of claim 52 wherein the degradable gemini surfactant is added in a range of about 0.1% to 20% by weight of the emulsified treatment fluid.

57. The subterranean treatment fluid of claim 52 wherein the emulsified treatment fluid further comprises a resin selected from the group consisting of an organic resin, an epoxy based resin, a novolak resin, a polyepoxide resin, a phenol-aldehyde resin, an urea-aldehyde resin, a urethane resin, a phenolic resin, a furan resin, a furan/furfuryl alcohol resin, a phenolic/latex resin, a phenol formaldehyde resin, a polyester resin, a polyurethane resin, acrylate resins, a hybrid and a copolymer of any of these, and any combination thereof.

58. The subterranean treatment fluid of claim 42 wherein the emulsified treatment fluid further comprises additives selected from the group consisting of a salt, a soap, a co-surfactant, a carboxylic acid, an acid, a fluid loss control additive, a gas, a foamer, a corrosion inhibitor, a scale inhibitor, a catalyst, a clay control agent, a biocide, a friction reducer, an antifoam agent, a bridging agent, a dispersant, a flocculant, an H2S scavenger, a CO₂ scavenger, an oxygen scavenger, a lubricant, a viscosifier, a breaker, a weighting agent, a relative permeability modifier, a resin, a particulate material, a wetting agent, a coating enhancement agent, and any combination thereof.

59. The subterranean treatment fluid of claim 52 wherein the emulsified treatment fluid is foamed with a gas.

60. The foamed treatment fluid of claim 52 wherein the gas is added in the range of about 5% to about 98% by volume of the foamed emulsified treatment fluid.
